(19) United States
(12) Patent Application Publication
(10) Pub. No.: US 2007/0255159 A1
(43) Pub. Date: Nov. 1, 2007

(54) Independent control and regulation of blood gas, pulmonary resistance, and sedation using an intravascular membrane catheter

(76) Inventors: Robert Quin-Yew Tham, Middleton, WI (US); Michael Mitton, San Prairie, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/412,282

(22) Filed: Apr. 27, 2006

(51) Int. Cl. A61B 5/08

(52) U.S. Cl. 600/529

(57) ABSTRACT

The present invention is a system and method of independent control and regulation of blood gas, sedation, and pulmonary resistance using an intravascular membrane catheter. The system and method monitors a patient to determine whether the patient requires an adjustment of his level of sedation, pulmonary vascular resistance, or some other pre-determined physiological blood concentration that can be controlled by adjusting metabolic and inhaled anesthetic gas concentrations in the blood. A gas mixer or blender combines the O₂ with the required gases, and the IGEC is reset to administer the gas mixture. To add an inhaled anesthetic such as desflurane, enflurane, halothane, isoflurane, sevoflurane, or some other anesthetic known in the art, the blended gases are directed through an anesthetic vaporizer, for example the TEC 5, TEC 6, TEC 7, or the Aladin™ vaporizer which are commercialized by GE Healthcare. To adjust the level of sedation, the concentration setting of the vaporizer can be adjusted to add the appropriate concentration of inhaled anesthetic agent to the gas mixture. Typically less than one or two percent of inhaled anesthetic concentration is sufficient to provide adequate patient sedation. In the case of the adjusting pulmonary vascular resistance, the O₂ will be mixed with an appropriate amount of CO₂ and administered to the patient.

### FIELD OF THE INVENTION

The present invention relates to the field of intravascular membrane catheters. More specifically, the present invention relates to the field of blood gas, sedation and pulmonary resistance regulation.

### BACKGROUND OF THE INVENTION

An Intravascular Gas Exchange Catheter (IGEC), which in effect is an artificial lung assist device, consists of a multi-lumen catheter with a cylindrical bundle of micro porous hollow fiber membranes woven into a mat at the end. The catheter is placed within the central venous blood stream in the primary vein that returns blood to the heart. Once inserted, oxygen gas flows from outside the patient, through the catheter and through the hollow fibers. As blood passes over the fibers, oxygen diffuses into the blood stream from the fibers, while carbon dioxide diffuses out of the blood stream into the fibers. Excess O2 and CO2 are removed back through the catheter out of the body. The device is inserted percutaneously via the femoral vein. A sutureless securement system with anti-microbial agents is then used to hold the catheter in place. The catheter fibers and components are coated with heparin to prevent coagulation.

Operation of IGECs has been discussed in prior-art literature. In particular, IGECs in U.S. Pat. No. 4,850,958 (apparatus for extra-pulmonary blood gas exchange) and U.S. Pat. No. 5,219,326 (inflatable percutaneous oxygenator). In these patents, as well as in U.S. Pat. No. 5,501,663 (inflatable percutaneous oxygenator with traverse hollow fibers) and U.S. Pat. No. 5,207,640 (method of anesthetizing a patient), percutaneous oxygenators are described to exclusively use oxygen in order to optimize blood oxygenation.

For example, the Hattler catheters (U.S. Pat. No. 4,911,689, U.S. Pat. No. 5,865,789, U.S. Pat. No. 5,336,164) use only oxygen to flow into the catheter tube to enable gas exchange in the hollow membrane fibers. As a consequence of using pure O₂ to the gas exchange catheter, CO₂ is eliminated without control.

### SUMMARY OF THE INVENTION

The present invention is a system and method of independent control and regulation of blood gas, sedation, and pulmonary resistance using an intravascular membrane catheter. The system and method monitors a patient to determine whether the patient requires an adjustment of his level of sedation, pulmonary vascular resistance, or some other pre-determined physiological blood concentration that can be controlled by adjusting metabolic and inhaled anesthetic gas concentrations in the blood. A gas mixer or blender combines the O₂ with the required gases, and the IGEC is reset to administer the gas mixture. To add an inhaled anesthetic such as desflurane, enflurane, halothane, isoflurane, sevoflurane, or some other anesthetic known in the art, the blended gases are directed through an anesthetic vaporizer, for example the TEC 5, TEC 6, TEC 7, or the Aladin™ vaporizer which are commercialized by GE Healthcare. To adjust the level of sedation, the concentration setting of the vaporizer can be adjusted to add the appropriate concentration of inhaled anesthetic agent to the gas mixture. Typically less than one or two percent of inhaled anesthetic concentration is sufficient to provide adequate patient sedation. In the case of the adjusting pulmonary vascular resistance, the O₂ will be mixed with an appropriate amount of CO₂ and administered to the patient.

One aspect of the present invention is a method of regulating blood gas with an intravascular gas exchange catheter (IGEC), the method comprising collecting a monitoring sample of respiratory parameters from a patent, mixing an additional gas with oxygen in a mixer to form a gas mixture when a pre-determined patient condition is detected and resetting the IGEC to administer the gas mixture to the patient, wherein resetting the IGEC controls an amount of oxygen that is added to the bloodstream of the patient and further remedies the pre-determined patient condition.

The resetting step may be effectuated manually by a user. The collecting of the monitoring sample of respiratory parameters is effectuated by a carestation, which includes devices capable of delivering blended gases, patient ventilation, monitoring respiratory mechanics and gas, and patient vital signs, for example pulse oximetry, electrocardiogram, cardiovascular circulatory parameters such as blood pressures and vascular resistances, depth of sedation monitor, and chart and record information, all are available from GE Healthcare product portfolio such as offered in the Aestiva™ and Aisys™ anesthesia machines, the Engstrom™ ventilator, and S/5 Anesthesia or Critical Care Monitor, and integrating the carestation with the IGEC, and the resetting step may be effectuated automatically when the carestation sends an instruction signal to the IGEC. The collecting step may be periodically activated when a user sets the carestation to an auto setting. The method further comprises setting the IGEC to a starting level based on a set of patient physiological data and further comprises setting the predetermined acceptable range on the carestation based on a set of patient physiological data. The IGEC is inserted into the patient through the femoral vein. The carestation includes a critical care ventilator, a respiratory monitor, patient cardiovascular monitor, and an information management system. When the predetermined patient condition is a measure of pulmonary vascular resistance not within a pre-determined range, an amount of carbon dioxide is mixed with oxygen in the mixing step. The carbon dioxide from the mixed gases reduces the rate of carbon dioxide washout from the blood thereby controllably affecting the pulmonary resistance.

Another aspect of the present invention is a system of regulating blood gas of a patient, the system comprising a mixer, configured to mix oxygen with an additional gas to form a gas mixture when a pre-determined patient condition is detected, an intravascular gas exchange catheter (IGEC) coupled to the mixer and inserted into the bloodstream of the patient and a controller, coupled to the mixer and configured to control the mixer and the IGEC, such that the appropriate gas mixture is added to the blood stream of the patient. Adjusting the IGEC controls an amount of oxygen that is added to the bloodstream of the patient and an amount of carbon dioxide removed from the bloodstream of the patient. The IGEC may be adjusted manually by a user using the controller. The IGEC is adjusted automatically by a carestation coupled to the mixer, when the carestation sends an instruction signal to the IGEC. The carestation collects the monitoring sample periodically when a user sets the carestation to an auto setting. The IGEC is set to a starting level based on a set of patient physiological data. The carestation is set to the predetermined acceptable range based on a set of patient physiological data, and the IGEC is inserted into the patient through the femoral vein. The carestation includes a critical care ventilator, a respiratory monitor and an information management system. The gas mixture includes oxygen and carbon dioxide when the pre-determined patient condition is a measure of pulmonary vascular resistance.

Another further aspect of the present invention is a method of regulating the predetermined level of patient sedation with an intravascular gas exchange catheter (IGEC) and a carestation, the method comprising coupling the carestation with the IGEC, collecting a monitoring sample of respiratory gas concentration, or measured patient sedation level using the Bispectral or Entropy Index measurement, either of which are available as a module of the patient monitor of the carestation, or another known method in the art, mixing a gas mixture with a mixer and adding inhaled anesthetic to the mixture with a vaporizer when a pre-determined patient condition is detected, and resetting the IGEC to administer the gas and inhaled anesthetic mixture to the patient, wherein adjusting the IGEC controls an amount of oxygen that is added to the bloodstream of the patient and further remedies the pre-determined patent condition.

Yet another aspect of the present invention is a method of regulating blood gas in a patient with an intravascular gas exchange catheter (IGEC) and a carestation, the method comprising coupling the carestation with the IGEC, collecting a monitoring sample of respiratory parameters with the carestation, mixing a gas mixture with a mixer when a pre-determined patient condition is detected; and resetting the IGEC to administer the gas mixture to the patient, wherein adjusting the IGEC controls an amount of oxygen that is added to the bloodstream of the patient and further remedies the pre-determined patent condition.

### BRIEF DESCRIPTION OF THE DRAWINGS

FIG. 1 illustrates a block diagram according to an embodiment of the present invention.

FIG. 2 illustrates a block diagram according to an embodiment of the system of the present invention.

FIG. 3 illustrates a flow chart depicting an embodiment of the method of the present invention.

### DETAILED DESCRIPTION OF THE INVENTION

The prior-art IGEC literature describes methods to operate an IGEC as a stand-alone device. However, the prior art does not describe the use of the IGEC to independently control other blood gas concentrations such as PCO₂ to influence the pulmonary vascular resistance, or inhaled anesthetics on patient sedation while oxygenating blood circulation. Such independent control and regulation is desired to improve blood oxygenation with an IGEC while concommittally improving the physiological well-being of the patient.

It is known that CO₂ in the pulmonary vessels alters pulmonary resistance. It is therefore desirable to control PCO₂ in the blood stream. In general, the demand for additional oxygenation in the blood stream dominates the choice of total flow rate into the hollow membrane fibers, however, CO₂ can and preferably be added by setting a CO₂ concentration of gas to the exchange catheter using a gas blender or mixer. The CO2 in the exchange catheter reduces the exchange gradient from the bloodstream causing the blood to retain CO2, which result in a higher PCO2.

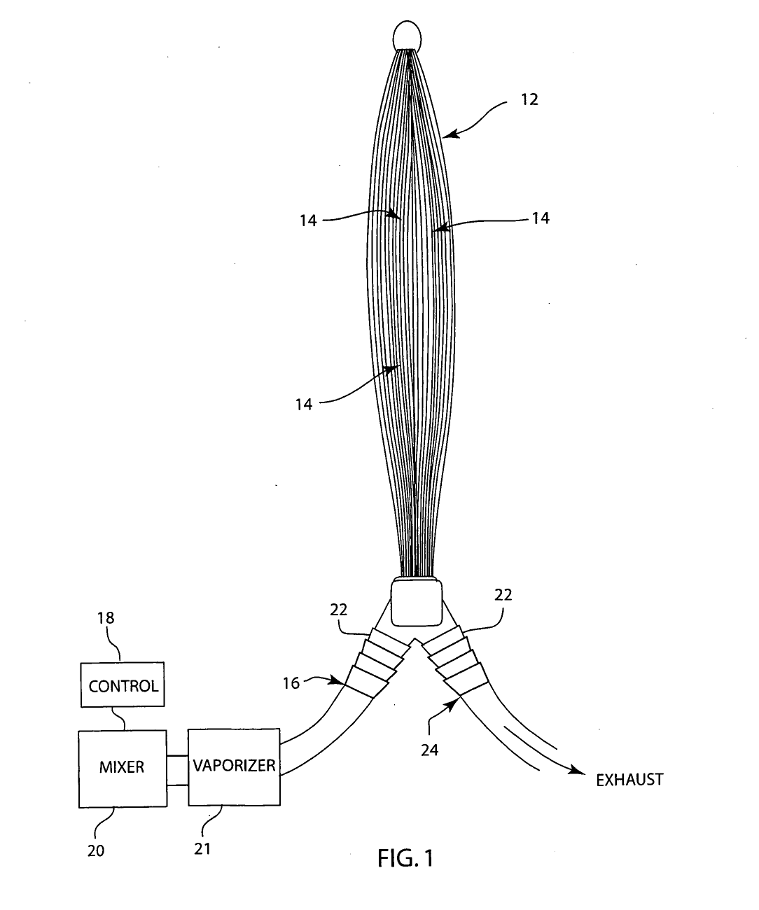

FIG. 1 depicts an embodiment of an IGEC 12. As described above, and in the cited references, a typical IGEC 12 includes a number of permeable fibers 14 that allow oxygen to be circulated through the IGEC 12, in order to oxygenate the blood of a patient. The IGEC 12 in FIG. 1 further includes a set of gas valves 22, that allows gas to enter the IGEC 12 through a gas inlet 16, and further allows gas to leave the IGEC 12 through the exhaust outlet 24. The gas inlet 16 of the IGEC 12 in FIG. 1 is coupled to a mixer 20, that allows a user to mix a gas or a plurality of additional gasses with the oxygen that is ordinarily circulated through an IGEC 12. The types and amounts of additional gases that are mixed in the gas mixer 20 are regulated by the control 18. Preferably, the control 18 is adjusted by a user, such as a physician or other caregiver. However, embodiments of the present invention are contemplated that allow a carestation monitoring the patient to automatically adjust the control 18 to create the appropriate gas mixture in the gas mixer 20. The gas mixture is then administered to the patient from the gas mixer 20, through the gas inlet 16 and further through the gas valve 22. The gas mixture is added to the blood stream through the permeable fibers 14, and any unused gas or collected gas from the blood stream leaves the IGEC 12 through the exhaust outlet 24. Preferably, this IGEC 12 allows a physician to add PCO₂ to the oxygen in order to influence the patients pulmonary vascular resistance, while oxygenating the blood. However, any desired mixture of gases be administered through the IGEC 12.

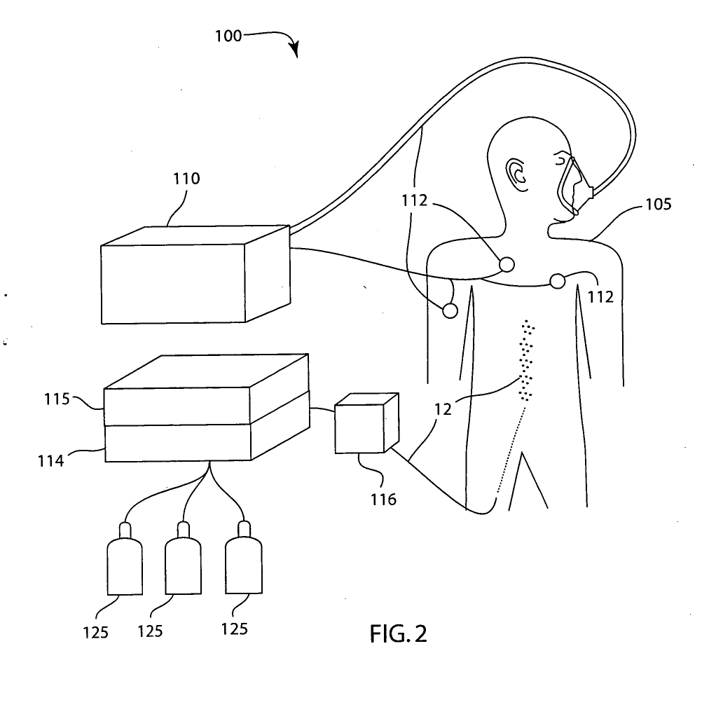

The IGEC system 100 of the present invention is depicted in FIG. 2. In FIG. 2, a patient 105 is monitored by a carestation 110, utilizing a number of physiological sensors 112, as required to collect the various physiological parameters set as patient waveforms, trends, gas monitoring, including inspired and expired O₂ and CO₂ concentrations, end title CO₂ (ETCO₂), CO₂ production and O₂ consumption, metabolic and energy expenditure, as well as patient spirometry. The carestation 110 collects this information from the patient 105 and displays the information for a user of the IGEC system 100, such as a caregiver or physician. The user may then adjust the IGEC control 115 accordingly, so that the patient 105 may receive the appropriate amount of blood oxygenation from the IGEC 12. As described above, the user activates a gas mixer 114 in order to mix the desired gas mixture from the gas supply 125 to be administered to the patient through the IGEC 12. In a preferred embodiment, the gas mixer 114 is operated through the controller 115, but other embodiments may include a gas mixer 114 having separate controls. The IGEC system 100 is also configured such that the user may create any mixture of gases necessary to influence a specific physiological parameter. The gas supply 12 will include all of the required gases, and may be configured as a plurality of gas tanks, or as a gas system that is permanently installed in a facility.

Still referring to FIG. 2, the IGEC 12 is preferably inserted through the femoral vein of the patient 105, and operates as described above. The IGEC 12 is controlled by an IGEC control 115, and may be coupled to the carestation 110 for automatic control. In additional embodiments of the present invention, when the patient 105 is displaying parameters that require a mixed gas solution, the carestation 110 will detect this condition, and instruct the IGEC control 115 to mix the gas supply 125 and adjust the oxygenation through the IGEC 120 automatically, and as described previously, the IGEC system 100 will be configured to adjust the ventilator automatically, as well as the ventilator and IGEC 12 in combination in order to adjust the patients 105 physiological parameters to a desired range.

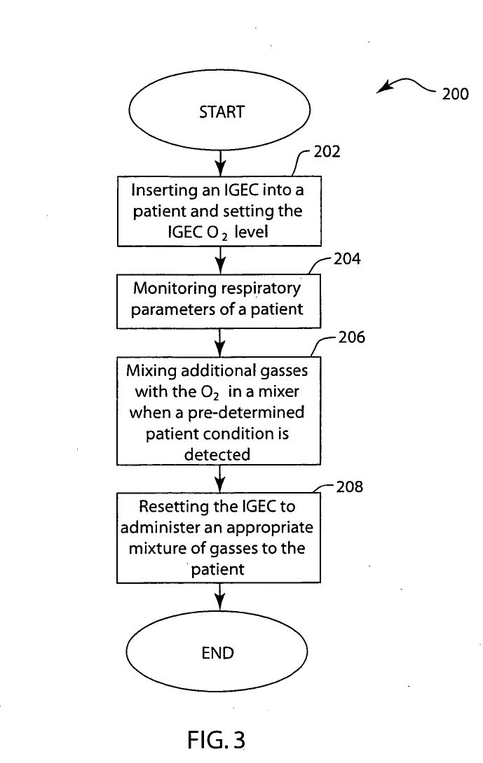

Referring now to FIG. 3, a control method 200 of the present invention is depicted. In step 202, an IGEC is inserted into a patient and set to a desired oxygenation level. In step 204, the patient respiratory parameters are monitored with the carestation. In step 206, appropriate gases are mixed with the oxygen in a mixer when a patient condition is detected by the carestation. A user preferably controls the gas mixture, and alternatively, the carestation adjusts the mixer automatically based on a preprogrammed set of criteria. In step 208, the IGEC is reset to administer the appropriate mixture of gases to the patient.

For patients with compromised pulmonary gas exchange and require an intravascular gas exchange catheter, this invention will provide an ability to control pulmonary vascular resistance through the addition of CO₂, or other desired gases, as a mixture of gases delivered to the gas exchange catheter.

Referring again to FIG. 1, for patients that require sedation during IGEC treatment, an anesthetic vaporizer 21 can be added downstream of the mixer 20 and regulated by the control 18. Gases from the mixer pass through the vaporizer 21 consisting of a split flow vaporizer that directs a portion of the gas flow through an anesthetic agent reservoir to pick up the concentration of anesthetic vapor. The split flow is regulated by the control 18 according to the user prescribed setting. It is well known in the anesthesia literature and commercial products that there are other methods of adding a settable, regulated concentration of inhaled anesthetic to a stream of gas flow. Likewise, gases from the outlet of the vaporizer 21 enters the IGEC 12 through a gas inlet 16, and further allows gas to leave the IGEC 12 through the exhaust outlet 24 as described previously. The resulting gas mixture is permeable to the membrane of the IGEC. Referring again to FIG. 2, the IGEC system 100 may also include a vaporizer 116 that would operate as described above.

The present invention has been described in terms of specific embodiments incorporating details to facilitate the understanding of the principles of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

We claim:

1. A method of regulating blood gas with an intravascular gas exchange catheter (IGEC) the method comprising:
a.) collecting a monitoring sample of respiratory parameters from a patent;
b.) mixing an additional gas with oxygen in a mixer to form a gas mixture when a pre-determined patient condition is detected; and
c.) resetting the IGEC to administer the gas mixture to the patient, wherein resetting the IGEC controls an amount of oxygen that is added to the bloodstream of the patient and further remedies the pre-determined patient condition.

2. The method of claim 1, wherein the resetting step is effectuated manually by a user.

3. The method of claim 1, wherein the collecting of the monitoring sample of respiratory parameters is effectuated by a carestation.

4. The method of claim 3, further comprising coupling the carestation with the IGEC, and wherein the resetting step is effectuated automatically when the carestation sends an instruction signal to the IGEC.

5. The method of claim 3, wherein the collecting step is periodically activated when a user sets the carestation to an auto setting.

6. The method of claim 1, further comprising setting the IGEC to a starting level based on a set of patient physiological data.

7. The method of claim 3, further comprising setting the predetermined acceptable range on the carestation based on a set of patient physiological data.

8. The method of claim 1, wherein the IGEC is inserted into the patient through the femoral vein.

9. The method of claim 3, wherein the carestation includes a critical care ventilator, a respiratory and patient vital signs monitor and an information management system.

10. The method of claim 1, wherein when the pre-determined patient condition is a measure of pulmonary vascular resistance not within a pre-determined range, an amount of carbon dioxide is mixed with oxygen in the mixing step.

11. The method of claim 1, further comprising adding a sedative in vapor form to the gas mixture when a pre-determined patient sedation level is detected.

12. A system of regulating blood gas of a patient, the system comprising:
a.) a mixer, configured to mix oxygen with an additional gas to form a gas mixture when a pre-determined patient condition is detected;
b.) an intravascular gas exchange catheter (IGEC) coupled to the mixer and inserted into the bloodstream of the patient; and
c.) a controller, coupled to the mixer and configured to control the mixer and the IGEC, such that the appropriate gas mixture is added to the blood stream of the patient,
wherein adjusting the IGEC controls an amount of oxygen that is added to the bloodstream of the patient and an amount of carbon dioxide removed from the bloodstream of the patient.

13. The system of claim 12, wherein the IGEC is adjusted manually by a user using the controller.

14. The system of claim 12, wherein the IGEC is adjusted automatically by a carestation coupled to the mixer, when the carestation sends an instruction signal to the IGEC.

15. The system of claim 14, wherein the carestation collects the monitoring sample periodically when a user sets the carestation to an auto setting.

16. The system of claim 12, wherein the IGEC is set to a starting level based on a set of patient physiological data.

17. The system of claim 14, wherein the carestation is set to the predetermined acceptable range based on a set of patient physiological data.

18. The system of claim 12, wherein the IGEC is inserted into the patient through the femoral vein.

19. The system of claim 14, wherein the carestation includes a critical care ventilator, a respiratory and patient vital signs monitor and an information management system.

20. The system of claim 12, wherein the gas mixture includes oxygen and carbon dioxide when the predetermined patient condition is a measure of pulmonary vascular resistance.

21. The system of claim 12, further comprising a vaporizer coupled to the mixer, the vaporizer configured to add a sedative to the gas mixture when a pre-determined patient sedation level is detected.

22. A method of regulating the sedation of a patient with an intravascular gas exchange catheter (IGEC), the method comprising:
a). collecting a monitoring sample of respiratory parameters with the carestation;
b.) adding a sedative to a gas mixture with a vaporizer when a pre-determined patient sedation level is detected; and
c.) resetting the IGEC to administer the gas mixture to the patient,
wherein adjusting the IGEC controls an amount of oxygen that is added to the bloodstream of the patient and further remedies the predetermined patent sedation level.
